(19) United States
(12) Patent Application Publication
(10) Pub. No.: US 2025/0051249 A1
(43) Pub. Date: Feb. 13, 2025

(54) PROCESS FOR CONVERTING WHOLE CRUDE TO ETHYLENE

(71) Applicants: LUMMUS TECHNOLOGY LLC, Houston, TX (US); Saudi Arabian Technology Company, Dhahran (SA); Chevron Lummus Global LLC, Richmond, CA (US)

(72) Inventors: Kandasamy Sundaram, Houston, TX (US); Essam Abdullah Al-Sayed, Houston, TX (US); Ronald Venner, Houston, TX (US); Kareemuddin Shaik, Houston, TX (US); Ujjal Mukherjee, Houston, TX (US); Mazin Tamimi, Houston, TX (US); Peter Kuzma, Houston, TX (US); Saud Al-Dulaijan, Houston, TX (US); Pedro Santos, Houston, TX (US); Ibrahim Abba, Houston, TX (US); Theodorus L.M. Maesen, Houston, TX (US); Khaled Al-Johani, Houston, TX (US); Bala Ramachandran, Houston, TX (US)

(73) Assignees: LUMMUS TECHNOLOGY LLC, Houston, TX (US); Saudi Arabian Technology Company, Dhahran (SA); Chevron Lummus Global LLC, Richmond, CA (US)

(21) Appl. No.: 18/797,042

(22) Filed: Aug. 7, 2024

Related U.S. Application Data
(60) Provisional application No. 63/518,278, filed on Aug. 8, 2023.

(51) Int. Cl. C07C 4/04; C10G 55/04

(52) U.S. Cl. CPC C07C 4/04; C10G 55/04; C10G 2300/1037; C10G 2400/20

(57) ABSTRACT

A process for converting whole crudes and other heavy hydrocarbon streams to produce ethylene. The process includes feeding a hydroprocessed medium boiling fraction, a hydroprocessed residue fraction, or both, and a light boiling fraction to a steam cracker and recovering a light fraction. The light fraction is fed to a first separator and recovering a C1-C3 stream and a mixed C4 and C4+ stream, comprising isoolefins, olefins, and dienes and feeding the mixed C4 and C4+ stream to a second separator and recovering a mixed C4 stream comprising isoolefins, olefins, and a C4+ stream. A portion of the mixed C4 stream is dimerized in a dimerization unit to produce a dimerized product stream, hydrogenated in a total hydrogenation system, producing a hydrogenated dimer product stream, and cracking the hydrogenated dimer product stream in a thermal cracking reactor system, producing one or more of hydrogen, methane, ethylene, propylene, n-butenes, and isobutene.

### FIELD OF THE DISCLOSURE

Embodiments herein relate to processes and systems for producing ethylene from crude oil and low-value heavy hydrocarbon streams.

### BACKGROUND

High-boiling compounds in crude oil may cause significant operational issues if they are sent to a steam cracker. High boiling compounds have a propensity to form coke due largely to their high asphaltene content. Therefore, the high boiling compounds are typically removed before sending the lighter fractions to different petrochemicals units, such as a steam cracker or an aromatic complex. However, the removal process increases the capital cost of the overall process and lowers profitability, as the removed high-boiling compounds can only be sold as low-value fuel oil. In addition, the conversion of vacuum residue without significant formation of HPNAs (heavy polynuclear aromatics) detrimental to steam cracker furnaces downstream of the process has been a challenge to date.

U.S. Pat. No. 3,617,493 describes a process in which crude oil is sent to the convection section of a steam cracker and then to a separation zone, where the portion of the feed boiling below about 450° F. is separated from the rest of the feed and then sent, with steam, into the high-temperature portion of the steam cracker and subjected to cracking conditions.

U.S. Pat. No. 4,133,777 teaches a process in which feed oil initially flows downwardly in trickle flow through a fixed bed of HDM catalysts, and then passes downwardly through a fixed bed of promoted catalysts containing selected GROUP VI and GROUP VIII metals, with very little hydrocracking occurring in this combination process.

U.S. Pat. No. 5,603,824 disclosed a process of upgrading a waxy hydrocarbon feed mixture containing sulfur compounds that boils in the distillate range, to reduce sulfur content and 85% point while preserving the high octane of naphtha by-products and maximizing distillate yield. The process employs a single, downflow reactor having at least two catalyst beds and an inter-bed redistributor between the beds. The top bed contains a hydrocracking catalyst, preferably zeolite beta, and the bottom bed contains a dewaxing catalyst, preferably ZSM-5.

U.S. Pat. No. 3,730,879 discloses a two-bed catalytic process for the hydrodesulfurization of crude oil or a reduced fraction, in which at least 50 percent of the total pore volume of the first-bed catalytic consists of pores in the 100-200 Angstrom diameter range.

U.S. Pat. No. 3,830,720 discloses a two-bed catalytic process for hydrocracking and hydrodesulfurizing residual oils, in which a small-pore catalyst is disposed upstream of a large-pore catalyst.

U.S. Pat. No. 3,876,523 describes a novel catalyst and a process for catalytically demetalizing and desulfurizing hydrocarbon oils comprising residual fractions. The process described therein utilizes a catalyst comprising a hydrogenation component, such as cobalt and molybdenum oxides, composited on an alumina. Although this catalyst is highly effective for demetalization of residual fractions and has good stability with time on stream, its utility is remarkably improved when this catalyst is employed in a particular manner in combination with a second catalyst having different critical characteristics. A catalyst of the type described in U.S. Pat. No. 3,876,523 will be referred as a first catalyst, it being understood that this first catalyst is to be situated upstream of the second catalyst having different characteristics.

U.S. Pat. No. 4,153,539 discloses that improved hydrogen utilization and/or higher conversions of the desired product are obtained in hydrotreating or hydrocracking processes when using amphora particles for hydrotreating of light hydrocarbon fractions, catalytic reforming, fixed-bed alkylation processes, and the like.

U.S. Pat. No. 4,016,067 discloses that hydrocarbon oils, preferably residual fractions, are catalytically hydroprocessed to very effectively remove both metals and sulfur and with particularly slow aging of the catalyst system by contacting the oil sequentially with two catalysts of different characteristics. The first catalyst, located upstream of the second catalyst, is characterized by having at least 60% of its pore volume in pores greater than 100 A. in diameter and other characteristics hereinafter specified. The second catalyst, located downstream with respect to the first catalyst, is characterized by having a major fraction of its pore volume in pores less than 100 A. in diameter.

The dual catalyst apparatus of U.S. Pat. No. 4,016,067 is used to demetallize and/or desulfurize any hydrocarbon oil that has metals and/or sulfur content-undesirably high for a particular application. The dual catalyst apparatus is particularly effective for preparing low metals and/or low sulfur content feedstocks for catalytic cracking or for coking. In the process to remove metals and sulfur, hydrocarbon oil also is concomitantly enriched in hydrogen, making it an even more suitable feedstock for either of these processes.

In general, these and other prior processes for converting whole crudes typically convert less than 50 percent of the crude to the more desirable end products, including petrochemicals such as ethylene, propylene, butenes, pentenes, and light aromatics, for example. Generally, 20 percent of the whole crude is removed upfront in processing, removing the heaviest components that are hard to convert. About another 20 percent of the whole crude is typically converted to pyrolysis oil, and about 10 percent is over-converted to methane. In some industries, it may be desired to further upgrade the end products.

For example, in order to meet the fuel blending requirements, such as octane rating or vapor pressure requirements, smaller olefin molecules, such as C1-C4s, may be upgraded to produce longer chain molecules. Alternatively, the smaller olefin molecules may be etherified so as to increase the oxygen content of the molecule and the resulting fuel blend. The etherification side reaction may also serve as a moderator reaction to control the main reactor, selectively forming dimers as opposed to trimers.

One commonly used method of upgrading smaller olefin molecules, such as C₂ to C₅ olefins, is a dimerization reaction. Mixed C4s, isobutene, isoamylenes, such as 2-methyl-1-butene and 2-methyl-2-butene, and pentenes, such as 1-pentene and 2-pentene, are commercially significant in many applications.

Dimerization reactions involve contacting an olefin with a catalyst in order to produce a long chain molecule. A dimer can consist of two or more constituent olefin molecules. For example, dimerization is a type of oligomerization reaction that is limited to a combination of only two olefin molecules. If the olefin feed contains only one type of olefin, a dimer product is formed. If the olefin feed contains two or more different olefins or olefin isomers, a codimer product may also be formed.

Specifically, isobutene olefin dimerization is widely used for producing isooctene, an intermediate that can be hydrogenated to produce isooctane, a high-value gasoline blending additive. Several representative olefin dimerization reactions are shown below:

A gas phase olefin dimerization process is disclosed in U.S. Pat. Nos. 3,960,978 and 4,021,502, where C₂ to C₅ olefins, fed as either pure olefins or in admixture with paraffins, are dimerized via contact with a zeolite fixed catalyst bed. Other dimerization processes are disclosed in, for example, U.S. Pat. Nos. 4,242,530, 4,375,576, 5,003,124, 7,145,049, 6,335,473, 6,774,275, 6,858,770, 6,936,742, 6,995,296, 7,250,542, 7,288,693, 7,319,180, 6,689,927, 6,376,731, 5,877,372, 4,331,824, 4,100,220 and U.S. Patent Application Publication Nos. 20080064911, 20080045763, 20070161843, 20060030741, 20040210093, and 20040006252, among others. Acid resin catalysts have also found use in various other petrochemical processes, including formation of ethers, hydration of olefins, esterifications, and expoxidations, such as described in U.S. Pat. Nos. 4,551,567 and 4,629,710.

Processes for dimerization of olefins over such resin catalysts require periodic shutdowns of the dimerization unit to replace and/or regenerate the catalysts. Further, such solid-catalyzed processes may require additives (“selectivators”) to promote the selectivity of the catalyst to the dimer, where the additives may result in unwanted acid throw, deactivating the catalyst, and may additionally require complicated separation processes to remove the additive from the resulting product streams.

### SUMMARY

In one aspect, embodiments disclosed herein relate to a process for converting whole crudes and other heavy hydrocarbon streams to produce ethylene. The process includes separating a whole crude into at least a light boiling fraction, a medium boiling fraction, and a high boiling residue fraction; processing the medium boiling fraction, the high boiling fraction, or both, in one or more hydroprocessing steps to produce a hydroprocessed medium boiling fraction, a hydroprocessed residue fraction, or both; feeding the hydroprocessed medium boiling fraction, hydroprocessed residue fraction, or both, and the light boiling fraction to a steam cracker; recovering a light fraction comprising C1-C₄ hydrocarbons and a pyrolysis oil; feeding the light fraction to a first separator and recovering a C1-C3 stream and a mixed C4 and C4+ stream, comprising isoolefins, olefins, and dienes; feeding the mixed C4 and C4+ stream, comprising isoolefins, olefins, and dienes to a second separator and recovering a mixed C4 stream comprising isoolefins, olefins, and a C4+ stream; dimerizing the mixed C4 stream in a dimerization unit to produce a dimerized product stream; hydrogenating the dimerized product stream in a total hydrogenation system, producing a hydrogenated dimer product stream; and cracking the hydrogenated dimer product stream in a thermal cracking reactor system comprising one or more thermal cracking reactors, producing one or more of hydrogen, methane, ethylene, propylene, n-butenes, and isobutene.

Other aspects and advantages will be apparent from the following description and the appended claims.

### BRIEF DESCRIPTION OF DRAWINGS

FIG. 1 is a simplified process flow diagram of a system for converting whole crudes and heavy hydrocarbons to produce ethylene according to embodiments herein.

FIG. 2 is an overall simplified process flow diagram of a system for converting whole crudes and heavy hydrocarbons to produce high-value products according to embodiments herein.

FIG. 3 is a simplified process flow diagram of a process for converting whole crudes and heavy hydrocarbons to produce ethylene according to embodiments herein.

### DETAILED DESCRIPTION

As used herein, the term “petrochemicals” refers to hydrocarbons including light olefins and diolefins, and C6-C8 aromatics. Petrochemicals thus refer to hydrocarbons including ethylene, propylene, butenes, butadienes, pentenes, pentadienes, as well as benzene, toluene, and xylenes. Referring to a subset of petrochemicals, the term “chemicals,” as used herein, refers to ethylene, propylene, butadiene, 1-butene, isobutene, benzene, toluene, and para-xylenes.

Hydrotreating is a catalytic process, usually carried out in the presence of free hydrogen, in which the primary purpose when used to process hydrocarbon feedstocks is the removal of various metal contaminants (e.g., arsenic), heteroatoms (e.g., sulfur, nitrogen, and oxygen), and aromatics from the feedstock. Generally, in hydrotreating operations cracking of the hydrocarbon molecules (i.e., breaking the larger hydrocarbon molecules into smaller hydrocarbon molecules) is minimized. As used herein, the term “hydrotreating” refers to a refining process whereby a feed stream is reacted with hydrogen gas in the presence of a catalyst to remove impurities such as sulfur, nitrogen, oxygen, and/or metals (e.g. nickel, or vanadium) from the feed stream (e.g. the atmospheric tower bottoms) through reductive processes. Hydrotreating processes may vary substantially depending on the type of feed to a hydrotreater. For example, light feeds (e.g. naphtha) contain very little and few types of impurities, whereas heavy feeds (e.g. ATBs) typically possess many different heavy compounds present in a crude oil. Apart from having heavy compounds, impurities in heavy feeds are more complex and difficult to treat than those present in light feeds. Therefore, hydrotreating of light feeds is generally performed at lower reaction severity, whereas heavy feeds require higher reaction pressures and temperatures.

Hydrocracking refers to a process in which hydrogenation and dehydrogenation accompany the cracking/fragmentation of hydrocarbons, e.g., converting heavier hydrocarbons into lighter hydrocarbons, or converting aromatics and/or cycloparaffins(naphthenes) into non-cyclic branched paraffin.

“Conditioning” and like terms as used herein refers to the conversion of hydrocarbons by one or both of hydrocracking and hydrotreating. “Destructive hydrogenation” and like terms refers to cracking of the hydrocarbon molecular bonds of a hydrocarbon, and the associated hydrogen saturation of the remaining hydrocarbon fragments, which can create stable lower boiling point hydrocarbon oil products and may be inclusive of both hydrocracking and hydrotreating.

“Dimerization” as used herein refers to a chemical reaction for upgrading smaller olefin molecules, such as C₂ to C₅ olefins to longer chain hydrocarbons. For an example, Isobutene is commercially significant in many applications and can be used as a feed for dimerization reactions. For example, isobutene is one of the comonomers in butyl rubber. Isobutene can also is dimerized to produce compounds that can be used as chemical feedstock for further reaction or in gasoline blending. Diisobutene, the isobutene dimer, is of particular commercial value in several applications. For example, diisobutene can be used as an alkylation reaction feedstock or as an intermediate in the preparation of detergents. Diisobutene can also be hydrogenated to pure isooctane (2,2,4-trimethyl pentane) which is highly preferred in gasoline blending.

Dimerization reactions involve contacting an olefin with a catalyst in order to produce a longer chain molecule. A dimer can consist of two constituent olefin molecules, while trimers can consist of three constituent olefin molecules. For example, dimerization is a type of reaction that is primarily limited to a combination of only two olefin molecules, producing a dimerized product with small amounts of trimers and longer chain molecules.

Specifically, C₄ olefin dimerization is widely used for producing isooctene, an intermediate that can be hydrogenated to produce isooctane, a high-value gasoline blending additive. A non-limiting example or representative olefin dimerization reaction is shown below:

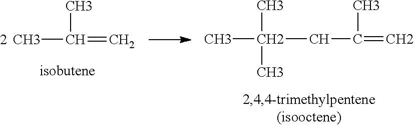

Hydrogenation as used herein relates to a catalytic chemical reaction where hydrogen is added to unsaturated hydrocarbons. One particular application of hydrogenation is to saturate olefins and diolefins to produce paraffin. The catalyst, for example, may be a metal catalyst such as platinum, palladium, or nickel. The hydrogenation reaction may be conducted at low temperatures in order to avoid cracking or other side reactions.

The integration of fractionation, hydroprocessing, steam cracking, dimerization, hydrogenation, and hydrocracking may result in a highly efficient facility, and in some embodiments may convert greater than 55%, greater than 60%, greater than 65%, greater than 70%, greater than 75%, greater than 80%, or greater than 85% of the whole crude to valuable petrochemicals, such as ethylene. In other embodiments, the integration of fractionation, hydroprocessing, steam cracking, dimerization, hydrogenation, and hydrocracking may result in a highly efficient facility, and in some embodiments may convert greater than 55%, greater than 60%, greater than 65%, greater than 70%, greater than 75%, greater than 80% or greater than 85% of the whole crude to small-chain hydrocarbons. Embodiments herein may thus provide systems and processes for conditioning feeds including even the heaviest, most undesirable residuum components into components that can be vaporized and passed into the radiant section of a steam cracker, substantially improving over the low petrochemical conversion of prior processes and reducing steam cracker fouling.

Embodiments herein relate to processes and systems that may take crude oil and/or low-value heavy hydrocarbons as feed and produce valuable petrochemicals including ethylene. More specifically, embodiments herein are directed toward process and systems for producing olefins by thermal cracking of a pre-conditioned crude oil or condensate, and using the olefins as feed for producing ethylene by processing through dimerization, hydrogenation, and hydrocracking. In one or more embodiments of processes disclosed herein may be to condition the residuum fraction of whole crude oils and condensates to produce additional feedstocks useful as a steam cracker feedstock. In another embodiment of processes disclosed herein may be to utilize the stream produced from steam cracker to further process and produce chemical streams including isoolefins. In other embodiments of processes disclosed herein may be to dimerize and hydrogenate isoolefins to produce ethylene as an end product.

Hydrocarbon mixtures useful in embodiments disclosed herein may include various hydrocarbon mixtures having a boiling point range, where the end boiling point of the mixture may be greater than 500° C., such as greater than 525° C., 550° C., or 575° C. The amount of high boiling hydrocarbons, such as hydrocarbons boiling over 550° C., may be as little as 0.1 wt %, 1 wt %, or 2 wt %, but can be as high as 10 wt %, 25 wt %, 50 wt % or greater. The description is explained with respect to crude oil, such as whole crude oil, but any high boiling endpoint hydrocarbon mixture can be used. However, processes disclosed herein can be applied to crudes, condensates, and hydrocarbon with a wide boiling curve and endpoints higher than 500° C. Such hydrocarbon mixtures may include whole crudes, virgin crudes, hydroprocessed crudes, gas oils, vacuum gas oils, heating oils, jet fuels, diesel, kerosenes, gasoline, synthetic naphthas, raffinate reformates, Fischer-Tropsch liquids, Fischer-Tropsch gases, natural gasoline, distillates, virgin naphthas, natural gas condensates, atmospheric pipe still bottoms, vacuum pipe still streams including bottoms, wide boiling range naphtha to gas oil condensates, heavy non-virgin hydrocarbon streams from refineries, vacuum gas oils, heavy gas oils, atmospheric residuum, hydrocracker wax, and Fischer-Tropsch wax, among others. In some embodiments, the hydrocarbon mixture may include hydrocarbons boiling from the naphtha range or lighter to the vacuum gas oil range or heavier.

When the end boiling point of the hydrocarbon mixture is high, such as over 550° C., the hydrocarbon mixture cannot be processed directly in a steam pyrolysis reactor to produce olefins. The presence of these heavy hydrocarbons results in the formation of coke in the reactor, where the coking may occur in one or more of the convection zone preheating coils or superheating coils, in the radiant coils, or in transfer line exchangers, and such coking may occur rapidly, such as in few hours. Whole crude is not typically cracked commercially, as it is not economical. It is generally fractionated, and only specific cuts are used in a steam pyrolysis heater to produce olefins. The remainder is used in other processes. The cracking reaction proceeds via a free radical mechanism. Hence, high ethylene yield can be achieved when it is cracked at high temperatures. Lighter feeds, like butanes and pentanes, require a high reactor temperature to obtain high olefin yields. Heavy feeds, like gas oil and vacuum gas oil (VGO), require lower temperatures. Crude contains a distribution of compounds from butanes to VGO and residue (material boiling over 550° C.). Subjecting the whole crude without separation at high temperatures produces a high yield of coke (a byproduct of cracking hydrocarbons at high severity) and plugs the steam pyrolysis reactor. The steam pyrolysis reactor has to be periodically shut down and the coke is cleaned by steam/air decoking. The time between two cleaning periods when the olefins are produced is called run length. When the whole crude is cracked without separation, coke can deposit in the convection section coils (vaporizing the fluid), in the radiant section (where the olefin producing reactions occur), and/or in the transfer line exchanger (where the reactions are stopped quickly by cooling to preserve the olefin yields).

Processes and systems for preparing feed stream for a steam cracker from the whole crude according to embodiments herein may include a feed preparation section, a crude conditioning section, an optional aromatic complex, and a steam cracker. The feed preparation section may include a desalter, for example. The desalted crude is then conditioned and processed such that crackable feed is being sent to the steam cracker and/or the aromatic complex. The conditioning section may allow an operator to maximize the yield of the chemical while maintaining a reasonable decoking frequency of the furnaces. Another objective of the crude conditioning unit is to ensure complete or essentially complete (95%+) conversion of asphaltenes to lower boiling point components that enhance the chemical's yield while reducing the formation of heavy polynuclear aromatics (HPNAs).

Processes according to embodiments herein may thus convert heavier fractions of crude oil into high-value petrochemicals and may minimize the amount of hydrocarbons sent to a fuel oil pool, which substantially increases profitability. The small fuel oil pool that is produced may also be upgraded into a low-sulfur, IMO 2020 compliant fuel oil, further increasing the value of the products.

As noted above, high-boiling compounds in the crude oil may cause significant operational issues if they are sent to a steam cracker, due to their propensity to form coke, mainly because of their high asphaltene content. Therefore, the high boiling compounds are typically removed before sending the lighter fractions to different petrochemicals units, such as the steam cracker and aromatic complex. The removal process increases the capital cost of the overall process and lowers profitability, as the removed high-boiling compounds can only be sold as low-value fuel oil. In addition, the conversion of vacuum residue without significant formation of HPNAs detrimental to steam cracker furnaces downstream of the process has been a challenge to date in the industry. Processes and systems according to embodiments herein may overcome these challenges.

The configurations of systems and processes for the conversion of whole crudes and heavy hydrocarbons according to embodiments described herein may efficiently handle residuals conversion while maximizing the petrochemicals conversion and maintaining lower coking propensity in the steam cracker. This is achieved by adding one or more integrated separation devices (ISD), solvent deasphalting units, and/or catalytic cracking units to the crude conditioning processes.

The upgraded crude streams from the crude conditioning unit, such as from a fixed bed crude conditioning unit and a hydrocracker, are suitable feedstocks for the steam cracker as well as an aromatic complex, when present. Such may lead to decreasing the overall process yields of low-value fuel oil and increasing the yields of high-value olefins.

Separation of various fractions, such as a low boiling fraction (a 160° C.− fraction, for example), a medium boiling fraction (a 160-490° C. fraction, for example), and a high boiling fraction (a 490° C.+ fraction, for example) may enhance the capital efficiently and operating costs of the processes and systems disclosed herein. While referring to three cuts in many embodiments herein, it is recognized by the present inventors that condensates, typically having a small amount of high boiling components, and whole crudes, having a greater quantity of high boiling components, may be processed differently. Accordingly, one, two, three, or more individual cuts can be performed for the wide boiling range of petroleum feeds, and each cut can be processed separately at optimum conditions.

Separation of the whole crude into the desired fractions may be performed using one or more separators (distillation columns, flash drums, etc.). In some embodiments, separation of the petroleum feeds may be performed in an integrated separation device (ISD), such as disclosed in US20130197283, which is incorporated herein by reference. In the ISD, an initial separation of a low boiling fraction is performed based on a combination of centrifugal and cyclonic effects to separate the desired vapor fraction from the liquid. An additional separation step may then be used to separate a middle boiling fraction from high boiling components.

Typically, hydrocarbon components boiling above 490° C. contain asphaltenes and Conradson Carbon Residue and thus need to be processed appropriately, as described further below. While embodiments are described as including a fraction below about 90° C.-250° C., such as a 160° C.− fraction, and a fraction above about 400° C.-560° C., such as a 490° C.+ fraction, it is noted that the actual cut points may be varied based on the type of the whole crude or other heavy fractions being processed. For example, for a crude containing a low metals or nitrogen content, or a large quantity of “easier-to-process” components boiling, for instance, at temperatures up to 525° C., 540° C., or 565° C., it may be possible to increase the mid/high cut point while still achieving the benefits of embodiments herein. Similarly, the low/mid-cut point may be as high as 220° C. in some embodiments, or as high as 250° C. in other embodiments. Further, it has been found that a low/mid-cut point of about 160° C. may provide a benefit for the sizing and operation of the reactors, such as a fixed bed conditioning reactor, for conditioning the mid fraction hydrocarbons (middle cut). Further still, for some feeds, such as condensate, the low/mid-cut point may be as high as 565° C. The ability to vary the cut points may add flexibility to process schemes according to embodiments herein, allowing for the processing of a wide variety of feeds while still producing the product mixture desired.

Accordingly, in some embodiments, the light cut may include hydrocarbons having a boiling point up to about 90° C. (e.g., a 90° C.− fraction), up to about 100° C., up to about 110° C., up to about 120° C., up to about 130° C., up to about 140° C., up to about 150° C., up to about 160° C., up to about 170° C., up to about 180° C., up to about 190° C., up to about 200° C., up to about 210° C., up to about 220° C., up to about 230° C., up to about 240° C., up to about 250° C. (e.g., a 250° C.− fraction), up to about 300° C., up to about 350° C., up to about 400° C., up to about 500° C., or up to about 565° C. Embodiments herein also contemplate the light cut being hydrocarbons having boiling points up to temperatures intermediate to the aforementioned ranges.

Depending upon the fractionation mechanism used, the light hydrocarbon “cut” may be relatively clean, meaning the light fraction may not have any substantial amount (>1 wt % as used herein) of compounds boiling above the intended boiling temperature target. For example, a 160° C.− cut may not have any substantial amount of hydrocarbon compounds boiling above 160° C. (i.e., >1 wt %). In other embodiments, the intended target “cut” temperatures noted above may be a 95% boiling point temperature, or in other embodiments as an 85% boiling point temperature, such as may be measured using ASTM D86 or ASTM D2887, or a True Boiling Point (TBP) analysis according to ASTM D2892, for example, and ASTM D7169 for heavy streams, such as those boiling above about 400° C. In such embodiments, there may be up to 5 wt % or up to 15 wt % of compounds above the indicated “cut” point temperature. For many whole crudes, the low/mid-cut point may be such that the light boiling fraction has a 95% boiling point temperature in the range from about 90° C. to about 250° C. For other feeds, however, such as condensate, the light boiling fraction may have a 95% boiling point temperature in the range from about 500° C. to about 565° C., for example.

In some embodiments, the middle cut may include hydrocarbons having a boiling point from a lower limit of the light cut upper temperature (e.g., 90° C., 100° C., 110° C., 120° C., 130° C., 140° C., 150° C., 160° C., 170° C., 180° C., 190° C., 200° C., 210° C., 220° C., 230° C., 240° C., 250° C., 300° C., 350° C., or 400° C., for example) to an upper limit of hydrocarbons having a boiling point up to about 350° C., up to about 375° C., up to about 400° C., up to about 410° C., up to about 420° C., up to about 430° C., up to about 440° C., up to about 450° C., up to about 460° C., up to about 480° C., up to about 490° C., up to about 500° C., up to about 520° C., up to about 540° C., up to about 560° C., or up to about 580° C., As used herein, for example, a middle cut having a lower limit of 160° C. and an upper limit of 490° C. may be referred to as a 160° C. to 490° C. cut or fraction. Embodiments herein also contemplate the middle cut being hydrocarbons having boiling points from and/or up-to temperatures intermediate to the aforementioned ranges.

Depending upon the fractionation mechanism, the hydrocarbon “cut” for the middle cut may be relatively clean, meaning the middle cut may not have any substantial amount (>1 wt %) of compounds boiling below and/or may not have any substantial amount (>1 wt %) of compounds boiling above the intended boiling temperature target limits. For example, a 160° C. to 490° C. cut may not have any substantial amount of hydrocarbon compounds boiling below 160° C. or above 490° C. In other embodiments, the intended target “cut” temperatures noted above may be a 5 wt % or 15 wt % boiling point temperature on the lower limit and/or a 95% or 85% boiling point temperature on the upper limit, such as may be measured using ASTM D86 or ASTM D2887, or a True Boiling Point (TBP) analysis according to ASTM D2892, for example, and ASTM D7169 for heavy streams, such as those boiling above about 400° C. In such embodiments, there may be up to 5 wt % or up to 15 wt % of compounds above and/or below the “cut” point temperature, respectively.

In some embodiments, the heavy cut may include hydrocarbons having a boiling point above about 350° C., above about 375° C., above about 400° C. (e.g., a 400° C.+ fraction), above about 420° C., above about 440° C., above about 460° C., above about 480° C., above about 490° C., above about 500° C., above about 510° C., above about 520° C., above about 530° C., above about 540° C., above about 560° C., above about 580° C., above about 590° C., above about 600° C. (e.g., a 600° C.+ fraction), or above about 700° C. Embodiments herein also contemplate the heavy cut being hydrocarbons having boiling points above temperatures intermediate to the aforementioned temperatures.

Depending upon the fractionation mechanism, the heavy hydrocarbon “cut” may be relatively clean, meaning the heavy fraction may not have any substantial amount (>1 wt %) of compounds boiling below the intended boiling temperature target. For example, a 490° C.+ cut may not have any substantial amount of hydrocarbon compounds boiling below 490° C. In other embodiments, the intended target “cut” temperatures noted above may be a 95% boiling point temperature, or in other embodiments as an 85% boiling point temperature, such as may be measured using ASTM D86 or ASTM D2887, or a True Boiling Point (TBP) analysis according to ASTM D2892, for example, and ASTM D7169 for heavy streams, such as those boiling above about 400° C. In such embodiments, there may be up to 5 wt % or up to 15 wt % of compounds, respectively, below the “cut” point temperature.

While examples below are given with respect to limited temperature ranges, it is envisioned that any of the temperature ranges prescribed above can be used in the processes described herein. Further, with respect to cut points, those referred to in the examples below may be clean, as described above, or may refer to 5% or 15% boiling temperatures for lower limits, or may refer to 85% or 95% boiling temperatures for upper limits.

Following fractionation, the light cut, such as a 160° C.− cut, may be fed to a steam cracker section of the system with or without further processing. The light cut fed to the steam cracker section may include light naphtha and lighter hydrocarbons, for example, and in some embodiments may include heavy naphtha boiling range hydrocarbons.

The mid-range hydrocarbon cut may be conditioned using one or more fixed bed reactors, such as hydrotreating and/or hydrocracking reactors, each of which may destructively hydrogenate the hydrocarbons in the mid-cut. The conditioning reactors may include catalysts for metals removal, sulfur removal, and nitrogen removal, and the conditioning in these reactors may overall add hydrogen to the hydrocarbon components, making them easier to process downstream to produce petrochemicals. The fixed bed catalyst systems in the mid-cut conditioning zone, for example, may contain different layers of demetalizing, destructive hydrogenation and mesoporous zeolite hydrocracking catalysts to optimize the conversion of the heavy materials to a balance between a highly paraffinic stream that is suitable for olefins production and a rich in aromatics stream that is suitable for aromatics production.

In some embodiments, it may be desirable to further separate the mid-cut into a low-mid cut and a high-mid cut. For example, a mid-cut having a boiling range from 160° C. to 490° C. may be divided into a low-mid cut having a boiling range from about 160° C. to about 325° C. and a high-mid cut having a boiling range from about 325° C. to about 490° C. The conditioning trains may thus be configured to more selectively convert the hydrocarbon components in the respective low and high mid cuts to the desired conditioned effluents, where each train may be configured based on preferred catalysts to destructively hydrogenate the hydrocarbons therein, reactor sizing for expected feed volumes and catalyst lifetime, as well as operating conditions to achieve the desired conversions to naphtha range containing steam cracker feedstocks. Similarly, division of the mid-cut into three or more sub-cuts is also contemplated.

Processing of the heavy hydrocarbons, such as 490° C.+ hydrocarbons, in the solvent deasphalting unit, may enhance the conversion of low-value streams to high-value products. The solvent deasphalting unit may further provide benefits, such as the ability to match run lengths of conditioning reactors with the steam cracker, as well as to provide an ability to handle a broader range of feeds and different crudes, allowing an operator to tune the process. The resulting solvent deasphalted oil may then be further treated, conditioning the deasphalted oil for use in the steam cracker system. It is recognized, however, that the lifetime of hydrotreating and/or hydrocracking catalysts may be negatively impacted by heavier components, such as where the feed includes components boiling above 565° C., for example.

The crude conditioning section is designed to achieve four (4) goals. First, the crude conditioning section may be used to increase the concentration of paraffin and naphthenes in the crude. Second, the conditioning section may decrease the concentration of polynuclear aromatic hydrocarbons (PNAs) in the crude. Third, the conditioning section may reduce the final boiling point (FBP) of the crude to below 540° C. And, fourth, the conditioning section may minimize the vacuum residue fraction of the crude oil.

Embodiments herein, when conditioning the middle and heavy (deasphalted oil) fractions, may target conversion of the heavier hydrocarbons to form hydrocarbons lighter than diesel, for example. The hydrotreating and hydrocracking catalysts and operating conditions may thus be selected to target the conversion of the hydrocarbons, or the hydrocarbons in the respective fractions, to primarily (>50 wt %) naphtha range hydrocarbons. In one or more embodiments, hydrotreating and hydrocracking catalysts and operating conditions may thus be selected to target the conversion of the hydrocarbons, or the hydrocarbons in the respective fractions, to primarily (>50 wt %) steam crackable products. The use of catalysts and operating conditions in the conditioning section to target lighter hydrocarbon products may enhance the operability of the steam cracker and the production of chemicals.

In some embodiments, conditioning of the heavy cut, such as a 490° C.+ cut, may result in the conversion of at least 70 wt % of the compounds boiling more than 565° C. to lighter boiling compounds. Other embodiments may result in the conversion of greater than 75 wt %, greater than 80 wt %, or greater than 85 wt % of the compounds boiling more than 565° C. to lighter boiling compounds.

In some embodiments, conditioning of the middle cut, such as a 160° C. to 490° C. cuts, may result in the conversion of greater than 50 wt % of the hydrocarbons therein to naphtha range hydrocarbons. In other embodiments, conditioning of the middle cut may result in the conversion of greater than 55 wt %, greater than 60 wt %, or greater than 65 wt %, or greater than 70 wt % of the hydrocarbons therein to naphtha range hydrocarbons.

In some embodiments, collective conditioning of the middle cut and the heavy cut may result in an overall conversion of greater than 50 wt % of the hydrocarbons therein to naphtha range hydrocarbons. In other embodiments, conditioning of the middle cut and the heavy cut may result in the conversion of greater than 55 wt %, greater than 60 wt %, or greater than 65 wt % of the hydrocarbons therein to naphtha range hydrocarbons.

As a result of such initial separations and conditioning, feeds to the steam cracker may be fed, in some embodiments, directly to the steam cracker without further processing. The light cut, having preferred properties, including one or more of boiling point, API, BMCI, hydrogen content, nitrogen content, sulfur content, viscosity, MCRT, or total metals content, may be fed directly to the steam cracker following separations in some embodiments. Effluents from the middle cut conditioning may also be fed directly to the steam cracker according to embodiments herein. Likewise, effluents from the heavy cut conditioning may be fed directly to the steam cracker in some embodiments.

The conditioning of the respective fractions as described herein may allow for the steam cracker, even while processing multiple feeds of varying boiling point ranges, to run for an extended period of time. In some embodiments, the steam cracker may be able to run for an uninterrupted run length of at least three years; at least four years in other embodiments; and at least five years in yet other embodiments.

Further, the initial hydrocarbon cut points, reactor sizes, catalysts, etc. may be adjusted or configured such that a run time of the steam cracker operations and conditioning processes may be aligned. The catalysts, reactor sizes, and conditions may be configured such that a run time of the conditioning unit is aligned with the run time of the steam cracker. Catalyst volumes, catalyst types, and reaction severity may all play a role in determining conditioning unit run times. Further, the extent of conditioning of the heavier hydrocarbons in the crude may impact coking in the thermal cracker. To maximize plant uptime, embodiments herein contemplate the design and configuration of the overall system such that the conditioning system has a similar anticipated run time as the steam cracker for a given feedstock or a variety of anticipated feedstocks. Further, embodiments herein contemplate adjustment of reaction conditions (T, P, space velocity, etc.) in the conditioning section and/or the steam cracker based on a feedstock being processed, such that a run time of the conditioning section and the steam cracker is similar or aligned.

Alignment of run times may result in minimal downtimes, such as when a catalyst turnover in a conditioning reactor is conducted concurrently with decoking of the steam cracker. Where the conditioning system includes multiple reactors or types of reactors, alignment of the run times may be based on the expected steam cracker performance. Further, where a hydrotreater, for example, may have a significantly longer run time than a hydrocracker in the conditioning section, parallel reactor trains and/or bypass processing may be used such that the overall run times of the conditioning and steam cracking units may be aligned.

Bypass processing may include, for example, temporarily processing a 490° C.+ cut in a reactor that normally processes a lighter feedstock. The heavier feedstock is anticipated to have more severe conditions and shorter catalyst life, and thus temporarily processing the heavies in a mid-range hydrocarbons conditioning reactor during a heavies catalyst change may allow the whole crude feed to continue to be fed to the steam cracker, without a shutdown, while the heavies conditioning reactor catalyst is replaced. Configuration of the mid-range conditioning reactors may also take into account the anticipated bypass processing when designing the overall system for aligned run times.

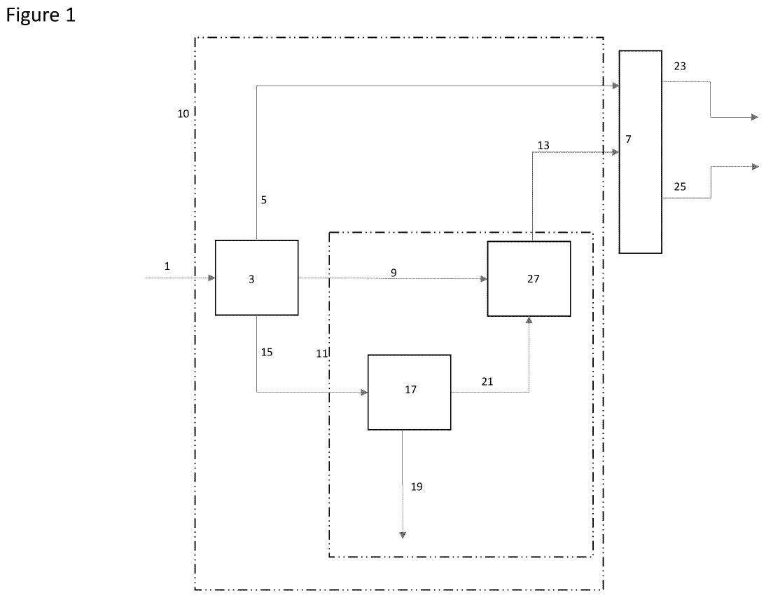

Referring now to FIG. 1, a simplified process flow diagram of a system for converting whole crudes and heavy hydrocarbons according to embodiments herein is illustrated.

In one or more embodiments, a desalted crude 1 may be treated in a feed processing system 10 to produce one or more chemical streams 23, and a higher boiling pyrolysis oil fraction 25.

As an example of the feed processing system 10, a wide boiling range of heavy hydrocarbon feed, such as a desalted crude 1, may be fed to a separation system 3. Separation system 3 may be an integrated separation device (ISD), as described above and including separation and heat integration, for example. In separation system 3, the desalted crude 1 may be separated into three fractions, including (a) a 160° C.− fraction 5 that doesn't require any conditioning and can be used as feed to the steam cracker section 7, (b) a 160-490° C. fraction 9 that may be upgraded in a conditioning section 11 to produce lighter hydrocarbons, such as a highly paraffinic stream 13 suitable for processing in the steam cracking section 7 and, (c) a 490° C.+ fraction 15, which contains the most refractory materials in the crude, and which can also be upgraded in the conditioning section 11 to produce lighter hydrocarbons, such as a highly paraffinic stream 13 suitable for processing in the steam cracking section 7. In one or more embodiments, the 490° C.+ fraction 15 is a solvent deasphalting system 17 in association with hydrotreaters and/or hydrocrackers 27. Other cut points may also be used to route the desired fractions and hydrocarbons therein to desired units for conditioning and/or cracking. The solvent deasphalting system 17 may produce a pitch 19 and a deasphalted oil 21 that is suitable to be further processed in the conditioning system to produce additional hydrocarbons suitable for conversion to chemicals in the steam cracker section 7. The processing of the feeds in the steam cracker section may produce one or more chemical streams 23, such as ethylene, propylene, and butenes, among others, as well as a higher boiling pyrolysis oil fraction 25. Other process schemes within feed processing system 10 may be used depending on the desired cut points, quality of the desalted crude feedstock, and desired products, among other factors.

In some embodiments, the mid-cut (160-490° C.) stream may be processed initially in a fixed bed conditioning system 27. The 490° C.+ stream may be processed in the solvent deasphalting system 17, which may include one or more solvent deasphalting units, to produce a deasphalted oil. The deasphalted oil may be further processed in the conditioning section 11, which may include the same reactor(s) used to condition the mid-cut, as illustrated, or a separate fixed bed conditioning system that may contain a catalyst tailored to effectively condition the deasphalted oil received from the solvent deasphalting system. The reaction products 13 from the fixed bed conditioning reactor 27 may then be co-processed in steam cracker section 7 for conversion into light olefins and other valuable chemicals. The pitch may be processed in one or more downstream processes (not illustrated).

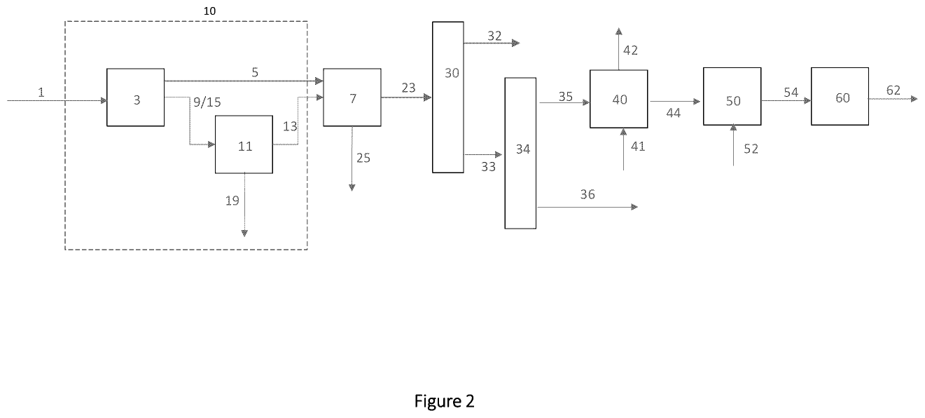

Referring now to FIG. 2, a simplified process flow diagram of a system for converting whole crudes and heavy hydrocarbons to produce ethylene according to embodiments herein is illustrated, where like numbers represent like parts.

A wide boiling range of heavy hydrocarbon feed, such as a desalted crude 1, may be fed to a feed processing system 10. Feed processing system 10 may include an integrated separation device (ISD) 3 as described above and including separation and heat integration, for example. In ISD 3, the desalted crude 1 may be separated into three fractions, including (a) a 160° C.− fraction 5 that does not require any conditioning and can be used as feed to the steam cracker section 7, (b) a 160-490° C. fraction 9 that may be upgraded in a conditioning section 11 to produce lighter hydrocarbons, such as a highly paraffinic stream 13 suitable for processing in a steam cracker 7 and, (c) a 490° C.+ fraction 15 that may also be upgraded in a conditioning section 11 to produce lighter hydrocarbons, such as a highly paraffinic stream 13 suitable for processing in a steam cracker 7. In addition to the highly paraffinic stream 13, the conditioning section 11 may also produce a pitch 19, which may contain the most refractory materials in the crude, and which can be further processed, or sent to the residual fuel oil pool as desired.

The conditioning section 11 may be utilized for processing the medium boiling fraction 9, the high boiling fraction 15, or both, in one or more hydroprocessing steps to produce the highly paraffinic stream 13 which may be a hydrotreated effluent stream, or a hydroprocessed medium boiling fraction stream, or a hydroprocessed residue fraction stream, or a combination thereof. Other cut points may also be used to route the desired fractions and hydrocarbons therein to desired units for conditioning, cracking, or both, as necessary. The processing of the highly paraffinic stream 13 in the steam cracker 7 may produce one or more chemical streams 23 such as ethylene, propylene, butenes, and other C4 and C4+ components, and a higher boiling pyrolysis oil 25. While not illustrated, the pyrolysis oil 25 may be recycled to the conditioning section 11 for further upgrading to chemicals 23.

The one or more chemicals stream 23 may be fed to a first separator 30 to recover a light fraction stream 32, which may include ethylene and propylene, and a heavy fraction 33, which may include butenes, isobutene, and other C4 and C4+ components including aromatics. The light fraction stream 32 may be recovered as a product, while the heavy fraction 33 may be fed to a second separator 34. The second separator 34 may separate the heavy fraction 33 into an overhead fraction 35 which may include the butenes, isobutenes, and other C4 components, and a bottoms fraction 36 including the C4+ components and aromatics. The bottoms fraction 36 may be further separated to recover an aromatic stream and a non-aromatic stream with the non-aromatic stream being recycled to one or more of the ISD 3, conditioning section 11, and steam cracker 7 for further upgrading to high-value products (not illustrated). The aromatic stream may be fed to the steam cracker 7 for further upgrading to high-value products (not illustrated).

The overhead fraction 35 may be sent to a dimerization reaction system 40. The dimerization reaction system 40 may contain several process units not specifically illustrated. For example, the dimerization reaction system 40 may include one or more separators, one or more reactors (which may be fixed bed, ebullated bed, or catalytic distillation reactors), and one or more internal recycle lines as known in the art. In one or more embodiments, the overhead fraction 35 sent to the dimerization reaction system 40 may be initially separated to recover an isoolefin fraction, such as an isobutene fraction. Hydrogen (H2) stream 41 may be fed to the dimerization reaction system, and the dimerization reaction system 40 may contain one or more reactors containing one or more catalysts configured to selectively hydrogenate any diolefins present in the overhead fraction 35, producing additional n-butenes, butanes, and isobutene. The one or more reactors containing one or more catalysts may use the H2 from the hydrogen stream 41 as the hydrogen source for the selective hydrogenation reaction.

The dimerization reaction system 40 may also contain one or more catalysts for dimerizing isobutene to for diisobutene as discussed previously. Following reaction in upstream reactors, such as fixed bed reactors, the effluent from the primary fixed bed reactor may be fed to a catalytic distillation column reactor to separate the reaction products while targeting complete conversion of the isobutene. Embodiments herein contemplate continued dimerization in the catalytic distillation column reactor.

After the isobutene in the overhead fraction, 35 is dimerized to produce diisobutene, the n-butenes and butanes present in the overhead fraction 35 may be separated from the dimerized product in the dimerization reaction system 40, produce a C4 product stream 42 and a dimerization product stream 44. The C4 product stream 42 may be recycled to one or more of the ISD 3, conditioning section 11, steam cracker 7, and first separator 30 for further upgrading to high-value products (not illustrated). In one or more embodiments, C4 product stream 42 may be recovered as a product and sent to one or more downstream process such as a metathesis process for conversion to propylene (not illustrated). While described with respect to isobutene dimerization, one of ordinary skill in the art will appreciate that other dimerization processes may be utilized.

The dimerization product stream 44, which may primarily contain diisobutene, may then be fed to a total hydrogenation system 50. The total hydrogenation system 50 may contain several process units not specifically illustrated. For example, the total hydrogenation system 50 may include one or more separators, one or more reactors (which may be fixed bed, ebullated bed, or catalytic distillation reactors), and one or more internal recycle lines as known in the art. The total hydrogenation system 50 may react the diisobutene in the dimerization product stream 44 with hydrogen 52 in the presence of a hydrogenation catalyst to form a hydrogenated product stream 54 containing isooctane.

The hydrogenated product stream 54 may then be sent to a thermal cracking reaction system 60. The thermal cracking reaction system 60 may contain several process units not specifically illustrated. For example, the thermal cracking reaction system 60 may include one or more separators, one or more reactors (which may be fixed bed, ebullated bed, or catalytic distillation reactors), and one or more internal recycle lines as known in the art. The isooctane in the hydrogenated product stream 54 may be thermally cracked in the thermal cracking reaction system 60 to produce one or more cracked products 62. These cracked products may include one or more of H2, methane, ethylene, propylene, n-butenes, isobutene, and other components.

The cracked products 62 may be separated and recovered as individual components within the hydrocracking reaction system 60 or may be sent as a whole product stream to a tail separation system (not illustrated). In either embodiment, a recovered H2 stream may be recycled to one or more hydrocracking reactors in the hydrocracking reaction system 60, recycled and combined with hydrogen stream 41, recycled and combined with hydrogen stream 52, or collected as product. The ethylene and propylene in the cracked products stream 62 may be combined with the ethylene and propylene in stream 32 and collected as high-value products. A portion of the n-butenes and isobutene may be recycled and combined with the overhead fraction 35 as additional feedstock or diluent for the dimerization process. A portion of the n-butenes and isobutene may also be combined with the C4 product stream 42 and sent to one or more downstream processes. Any components heavier than C4 which may be present in the cracked products 62 may be recycled to the chemicals stream 23 for additional upgrading or may be collected and sent to another downstream process as desired.

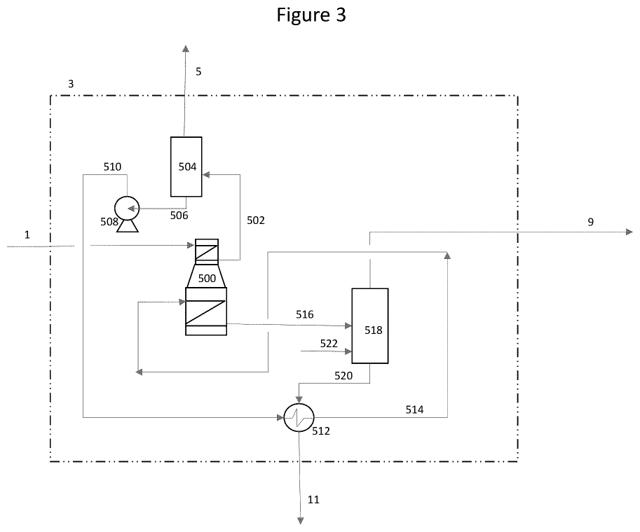

As described with respect to FIGS. 1 and 2, separation system 3 may be as illustrated in FIG. 3, including separation and heat integration. After desalting, the crude 1 may be further preheated in the convection section of a heater 500 to produce a preheated crude 502. The preheated crude 502 may be fed to a separator 504 which may facilitate the separation of the 160° C.− fraction 5 from heavier components, recovered in stream 506.

The remaining 160° C.+ crude fraction 506 may be fed to pump 508, which produces a pressurized 160° C.+ crude fraction 510, which may then be fed to a heat exchanger 512. ISD heat exchanger 512 may preheat the 160° C.+ crude fraction 510 against hot hydrogen stripper bottoms 520, producing a pressurized and pre-heated 160° C.+ crude fraction 514. The pressurized and pre-heated 160° C.+ crude fraction 514 may then be fed back to the heater 500, where it is heated to facilitate the separation of a 160-490° C. fraction from a heavier 490° C.+ fraction. The heated 160° C.+ crude fraction 516 may then be fed to a hot hydrogen stripper 518. In the hot hydrogen stripper 518, the 160° C.+ crude fraction is further separated into a 160-490° C. fraction 9 and the hot hydrogen stripper bottoms 520, which contains heavier 490° C.+ hydrocarbons. The hot hydrogen stripper bottoms 520, after being cooled via indirect heat exchange in heat exchanger 512 against the pressurized 160° C.+ crude fraction 510, may be removed from the separation system 3 as the 490° C.+ fraction 11.

The hot hydrogen stripper 518 may utilize a hydrogen feed 522 as the stripping medium. The hot hydrogen stripper 518 may be operated to provide broad flexibility, based on the nature of the crude feedstock that is being processed. The stripper overheads, which is the 160-490° C. fraction 9, may be cooled, to recover hydrogen, and routed to the intermediate hydroprocessing reaction stages as appropriate, and as described with respect to FIGS. 1 and 2. The recovered hydrogen may be fed to a downstream pressure swing adsorption (PSA) unit (not shown), after amine treatment (not shown), to improve the hydrogen purity. The PSA hydrogen product may be compressed in a make-up hydrogen compressor (not shown) to provide the make-up hydrogen for the one or more hydroprocessing reactors (FIG. 1), and as hot hydrogen feed 522.

The hot hydrogen stripper bottoms product 520 (such as a 490° C.+ cut) contains the most difficult compounds which must be handled in the crude, including asphaltenes, metals, and CCR. The excessive amount of metals, CCR, and asphaltenes in the high boiling residue fraction leads to rapid fouling of catalyst and an increase of pressure drop in fixed bed down-flow reactors, limiting both conversion and catalyst run length. After cooling against the pressurized 160° C.+ crude fraction 510, the 490° C.+ stream 11 may be recovered and processed in a liquid circulation, ebullated bed residue hydrocracker, as described in FIG. 1, along with any additional low-value refinery streams, such as a pyoil stream and/or slurry oil stream.

By adjusting the amount of hydrogen 522 fed to the hot hydrogen stripper 518, as well as the operating conditions of the hot hydrogen stripper 518 and heater 500, the hydrocarbon cut points may be adjusted such that the light-cut 5 may be fed directly to the downstream steam cracker, and the mid-cut 9 may have little to no deleterious compounds that would rapidly foul the fixed bed conditioning reactors. In this way, the separation system 3 (with the hot hydrogen stripper 518) may concentrate the most difficult to process hydrocarbons in the heavy-cut 11 which may be fed to the ebullated bed reactors which may be operated under the most severe conditions, thereby preserving the catalysts in the steam cracker and fix bed conditioning reactors.

As described above, embodiments herein may be used to convert heavier fractions of crude oil into high-value chemicals and may minimize the amount sent to a residual fuel oil pool, which increases profitability.

Unless defined otherwise, all technical and scientific terms used have the same meaning as commonly understood by one of ordinary skill in the art to which these systems, apparatuses, methods, processes, and compositions belong.

The singular forms “a,” “an,” and “the” include plural referents, unless the context clearly dictates otherwise.

As used here and in the appended claims, the words “comprise,” “has,” and “include” and all grammatical variations thereof are each intended to have an open, non-limiting meaning that does not exclude additional elements or steps.

“Optionally” means that the subsequently described event or circumstances may or may not occur. The description includes instances where the event or circumstance occurs and instances where it does not occur.

When the word “approximately” or “about” is used, this term may mean that there can be a variance in the value of up to ±10%, of up to 5%, of up to 2%, of up to 1%, of up to 0.5%, of up to 0.1%, or up to 0.01%.

Ranges may be expressed as from about one particular value to about another particular value, inclusive. When such a range is expressed, it is to be understood that another embodiment is from the one particular value to the other particular value, along with all particular values and combinations thereof within the range.

While the disclosure includes a limited number of embodiments, those skilled in the art, having the benefit of this disclosure, will appreciate that other embodiments may be devised which do not depart from the scope of the present disclosure. Accordingly, the scope should be limited only by the attached claims.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, any means-plus-function clauses are intended to cover the structures described herein as performing the recited function(s) and equivalents of those structures. Similarly, any step-plus-function clauses in the claims are intended to cover the acts described here as performing the recited function(s) and equivalents of those acts. It is the express intention of the applicant not to invoke 35 U.S.C. § 112(f) for any limitations of any of the claims herein, except for those in which the claim expressly uses the words “means for” or “step for” together with an associated function.

What is claimed:

1. A process for converting whole crudes and other heavy hydrocarbon streams to produce ethylene, the process comprising:
separating a whole crude into at least a light boiling fraction, a medium boiling fraction, and a high boiling residue fraction;
processing the medium boiling fraction, the high boiling fraction, or both, in one or more hydroprocessing steps to produce a hydroprocessed medium boiling fraction, a hydroprocessed residue fraction, or both;
feeding the hydroprocessed medium boiling fraction, hydroprocessed residue fraction, or both, and the light boiling fraction to a steam cracker;
recovering a light fraction comprising C1-C4 hydrocarbons and a pyrolysis oil;
feeding the light fraction to a first separator and recovering a C1-C3 stream and a mixed C4 and C4+ stream, comprising isoolefins, olefins, and dienes;
feeding the mixed C4 and C4+ stream, comprising isoolefins, olefins, and dienes to a second separator and recovering a mixed C4 stream comprising isoolefins, olefins, and a C4+ stream;
dimerizing the mixed C4 stream in a dimerization unit to produce a dimerized product stream;
hydrogenating the dimerized product stream in a total hydrogenation system, producing a hydrogenated dimer product stream; and
cracking the hydrogenated dimer product stream in a thermal cracking reactor system comprising one or more thermal cracking reactors, producing one or more of hydrogen, methane, ethylene, propylene, n-butenes, and isobutene.

2. The process of claim 1, wherein dimerizing comprises:
feeding the mixed C4 stream, comprising isoolefins, olefins, and dienes and a hydrogen stream to a first separator to separate the isoolefins and dienes from the olefins;
feeding the isoolefins and dienes to a selective hydrogenation unit to convert the dienes to olefins and isoolefins, producing a partially hydrogenated C4 stream;
feeding the partially hydrogenated C4 stream to a fixed bed reactor system containing a catalyst, producing a reactor effluent comprising dimers of the isoolefin and unreacted C4s;
feeding the reactor effluent to a product splitter, producing an overheads stream comprising unreacted C4 olefins and isoolefins, and a bottoms product comprising the dimerized product stream.

3. The process of claim 1, wherein separating the whole crude comprises:
feeding the whole crude into a heater, producing a pre-heated hydrocarbon feedstock;
separating the pre-heated hydrocarbon feedstock in a separator into the light boiling fraction and an intermediate fraction;
feeding the intermediate fraction back to the heater, producing a heated intermediate fraction;
feeding a hydrogen stream to a hot hydrogen stripper;
separating the heated intermediate fraction in the hot hydrogen stripper into the medium boiling fraction and a hot hydrogen stripper bottoms fraction; and
cooling the hot hydrogen stripper bottoms fraction via indirect heat exchange against the intermediate fraction producing the high boiling residue fraction.

4. The process of claim 1, the processing the high boiling fraction further comprises:
feeding the high boiling fraction in a solvent deasphalting unit to produce a deasphalted oil fraction and a pitch fraction.

5. The process of claim 4, further comprising destructively hydrogenating the medium boiling fraction and the deasphalted oil fraction to produce the hydroprocessed medium boiling fraction, hydroprocessed residue fraction, or both.

6. The process of claim 1, wherein hydrogenating the dimerized product stream in the hydrogenation unit comprising:
feeding the dimerized product stream to the total hydrogenation system along with a hydrogen stream to convert the dimers of the isoolefins in the dimerized product stream to paraffins, wherein the paraffins comprise isooctane.

7. The process of claim 2, further comprising recycling a portion of the unreacted C4 olefins and isoolefins to the selective hydrogenation unit, the dimerization unit, the hydrocracking reactor system, or a combination thereof.

8. The process of claim 1, wherein the thermal cracking further comprising processing the hydrogenated dimer product stream in a conditioning system comprising one or more thermal cracking reactors configured to thermally crack the hydrogenated dimer product stream to produce the one or more of hydrogen, methane, ethylene, propylene, n-butenes, and isobutene.

9. The process of claim 8, further comprising recycling a portion of the hydrogen to one or more hydrocracking reactors in the hydrocracking reaction system.

10. The process of claim 8, further comprising recycled a portion of the hydrogen to the dimerization unit as the hydrogen stream.

11. The process of claim 8, further comprising recycling a portion of the hydrogen to the total hydrogenation unit.

12. The process of claim 8, further comprising combining the ethylene and the propylene C1-C3 stream.

13. The process of claim 8, further comprising recycling a portion of the n-butenes and isobutene to the dimerization unit.
